(19) United States
(12) Patent Application Publication
(10) Pub. No.: US 2020/0241007 A1
(43) Pub. Date: Jul. 30, 2020

(54) METHOD FOR ESTIMATING THE EFFECTIVENESS AGAINST RHEUMATOID ARTHRITIS OF A T-LYMPHOCYTE CO-STIMULATION MODULATOR AGENT

(71) Applicant: SINNOVIAL, Grenoble (FR)

(72) Inventors: Anaïs COURTIER, Voiron (FR); Minh Vu Chuong NGUYEN, Le Versoud (FR); Athan BAILLET, Jarrie (FR); Philippe GAUDIN, Meylan (FR); Jacques-Eric GOTTENBERG, Strasbourg (FR)

(73) Assignee: SINNOVIAL, Grenoble (FR)

(21) Appl. No.: 16/756,600

(22) PCT Filed: Oct. 18, 2018
(86) PCT No.: PCT/FR2018/052605

§ 371 Date: Apr. 16, 2020

(30) Foreign Application Priority Data
Oct. 19, 2017 (FR) 1759861

(51) Int. Cl. G01N 33/68; G01N 33/564; G16B 25/10; G16C 20/30

(52) U.S. Cl. CPC G01N 33/6893; G01N 33/564; G16B 25/10; G01N 2800/52; G01N 2333/4737; G01N 2333/78; G01N 2800/102; G16C 20/30

(57) ABSTRACT

The disclosure relates to a method for estimating the effectiveness of treatment with a T-lymphocyte cell co-stimulation modulator agent in a patient with rheumatoid arthritis and who has had an inadequate response to prior biotherapy, consisting in analysing a biological sample of said patient for the expression of a set of biomarkers, the results of which make it possible to determine whether said modulator agent is a treatment that will engender a beneficial response for said patient. The disclosure also relates to a system for estimating the effectiveness of said treatment in said patient comprising means for measuring or receiving data concerning the expression level of said biomarkers and means for processing these data configured to estimate said effectiveness of said treatment in said patient. The biomarkers comprise at least two biomarkers selected from the group consisting of C4b-Binding Protein, C-Reactive Protein, Cartilage Oligomeric Matrix Protein, Fibronectin and Lipopolysaccharide-Binding Protein.

### FIELD OF THE INVENTION

The present invention relates to the treatment of rheumatoid arthritis. It more particularly relates to a method for estimating the effectiveness of treatment with a T-lymphocyte cell co-stimulation modulator agent, in particular with Abatacept (ABA), in a patient with rheumatoid arthritis (RA) and having had an inadequate response to one or more prior treatment(s) with biotherapy, consisting in analysing a biological sample of said patient vis-à-vis the expression of a set of biomarkers, the correlation of the results obtained for this set of biomarkers making it possible, notably by comparison with reference values, to determine if the T-lymphocyte cell co-stimulation modulator agent is a promising treatment making it possible to lead to a beneficial response for said patient.

The present invention also relates to a system for estimating the effectiveness of said treatment in said patient comprising means for measuring or receiving data of the expression level of said biomarkers and means for processing these data configured to estimate said effectiveness of said treatment in said patient.

### BACKGROUND OF THE INVENTION

Rheumatoid arthritis (RA) is a chronic inflammatory disease characterised by synovitis, joint injuries, functional handicap and a significant increase in mortality.

Early intervention using sDMARDs (synthetic Disease-Modifying Anti-Rheumatic Drugs) is now recognised as being essential for preventing structural joint damage and progressive loss of function (Smolen et al., 2017). For patients that do not respond to treatment with sDMARDs or who develop an inadequate response to these drugs over time, bDMARDs (biological DMARDs) are one effective additional treatment option (Smolen et al., 2017). In clinical practice, the first choice of biological therapy is normally a TNF-a (Tumour Necrosis Factor alpha) inhibitor. Response to treatment can vary in a very significant manner from one patient to another. Around 30% to 40% of patients who start treatment with a TNF-a inhibitor develop thereafter either an insufficient response for ineffectiveness of the treatment, or undesirable events, with these drugs (Souto et al., 2016). The options for the continuation of treatment in TNF-a insufficient patients comprise the use of a second biological agent. Given the number of bDMARD treatment options available for clinicians and their effectiveness in the treatment of RA, the passage between different bDMARDs is common practice (Zhang et al., 2011). That is why, at the present time, the practitioner recommends, for most diseases, a first-line treatment then, in the event of insufficient or inadequate response, a second-line treatment, and so on. However, within this overall strategy, there exists a debate on the relative effectiveness of the use of an alternative inhibitor to TNF-a (cycle) or a biological agent with a different mode of action (switching). In addition, the probability for the patient having already received an anti-TNF of responding to another biological treatment decreases progressively as a function of the increasing number of failures of prior treatments (Rendas-Baum et al., 2011). Thus, data from the literature indicated that any early intervention makes it possible to better contain the progression of the disease.

Unfortunately, today, the practitioner lacks objective elements capable of helping him in his therapeutic choice in order to be able to target and to identify a priori the appropriate treatment for his patient. A tool capable of providing the clinician with a score of probability of response or non-response to a treatment would be an important beneficial element.

Indeed, the time lost bringing to light a potential therapeutic failure is to the detriment of the effectiveness of the therapeutic action and the well-being of the patient, which in certain cases may have ended up in new symptoms or prejudicial and irreversible consequences in terms of general condition. In addition, they may be costly treatments which may be cumbersome to put in place, which is entirely unsatisfactory when a therapeutic failure is noted.

Considerable progress has been made over recent years concerning the diagnosis, care, treatment and follow-up of patients with chronic inflammatory diseases.

In terms of treatments of chronic inflammatory diseases, and notably chronic inflammatory rheumatisms, biotherapies notably exist which consist of biological molecules, such as proteins, antibodies, having a therapeutic action. Some of them are already used and others are under development.

Among chronic inflammatory diseases, rheumatoid arthritis is an auto-immune disorder of the synovia which is characterised by the proliferation of synoviocytes and the infiltration of inflammatory cells into the joint. Various cytokines play an important role in the regulation of inflammatory diseases.

bDMARDs targeting the tumoral necrosis factor (TNF) or the co-stimulation of T-lymphocyte cells for example have enabled considerable progress for the treatment of RA. At present, 9 bDMARDs including the T-lymphocyte cell co-stimulation modulator Abatacept (ABA), anti-IL-6 Tocilizumab (TCZ), anti-CD20 Rituximab (RTX), anti-Interleukine-1 (IL-1) Anakinra (ANK) and anti-TNF-a Adalimumab (ADA), Etanercept (ETN), Infliximab (IFX), Golimumab (GOL) and Certolizumab Pegol (CTP), are approved for the treatment of rheumatoid arthritis. However, the responses to each biological agent vary for each individual. Consequently, making an optimal choice of one or more bDMARD(s) within a therapeutic window of opportunity is essential to obtain effectiveness of treatment, which proves to be very expensive. Indeed, the chances of success of a biological treatment dwindle as a function of the increasing number of therapeutic failures with biotherapy (Rendas-Baum et al., 2011).

The practitioner lacks however elements at his disposal to help him in his therapeutic choice. A tool capable of providing the clinician with a score of probability of response or non-response to a treatment would certainly be welcome.

In particular, there lacks at present very early biomarkers which can, among other things, provide guidance with regard to the possible response or non-response to a biological or conventional background treatment.

These biomarkers call on molecular biology and biochemistry. The hypothetico-deductive approach has reduced personalised medicine to several biomarkers, the interest of which has been fixed a priori and which has not made it possible to exhaust questions of early diagnosis or the theranostic approach. Thus, the search for THE biomarker making it possible to predict THE response to a biological treatment in chronic inflammatory diseases, and thus in RA, is an illusion. The multiplicity of genetic or biochemical biomarkers associated with the good clinical response or non-response to a biological treatment makes the task difficult.

Genomics, transcriptomics, epigenetics and proteomics are complementary and non-redundant pillars in this perspective.

In genomic terms (Prajapati et al., 2011), the replication of results on different cohorts is fragile. The hypothesis of several associated genes each having a small impact is favoured compared to that where few genes each with an important effect could be involved. Its routine use remains complex.

Epigenetics has also provided data, for the moment very preliminary (Krintel et al., 2016).

The study of the transcriptome makes it possible to identify a certain number of genes, the multiplicity of which makes use on a daily basis difficult (Smith et al., 2013).

The study of the serum metabolome remains complicated (Tatar et al., 2016).

Finally, for about 10 years now the proteomic approach has been real in Rheumatology for theranostic ratings (Trocmé et al., 2009).

This predictive approach of personalised or stratified medicine type is very innovative in the field of chronic inflammatory rheumatisms and could make it possible to prescribe the right treatment to the right patient at the right moment, to limit the progression of the handicap by guiding the patient as quickly as possible to the treatment to which he has the greatest chance of responding, and to avoid prescribing treatments which, conversely, are associated with a low probability of response.

Abatacept is a fusion protein composed of the modified Fc region of human immunoglobulin (IgG1) fused to the extracellular domain of the protein CTLA-4 (Cytotoxic T-lymphocyte Associated Protein 4, also called CD152). Abatacept selectively modulates the key co-stimulation signal required for the complete activation of T-lymphocytes expressing the receptor CD28 (Cluster of Differentiation 28). In order that a T-cell is activated and induces an immune response, an antigen presenting cell must present two signals to the T-cell. The first signal is the recognition of a specific antigen by a T-cell receptor and the second signal is the co-stimulation by the bond of the molecules CD80 or CD86 (also known as B7-1 and B7-2) to the receptor CD28 on the T-cells. Abatacept selectively inhibits this co-stimulatory pathway by specifically binding to CD80 and CD86, thereby preventing the second signal from being set up. It thus inhibits the complete activation of the T-cells.

Some studies have focused on highlighting biomarkers making it possible to predict the response to ABA treatment in patients with RA. These studies mainly concern the characterisation of biomarkers of DNA and RNA (Derambure et al., 2017; Nakamura et al., 2016) or even of specific circulating cells (Scarsi et al., 2011). However, DNA and RNA are subjected to potential modifications (epigenetic, regulation of the expression of the genes, splicing) linked to the environment before being translated into proteins which are the final effectors. The proteomic approach thus makes it possible to minimise the possible variations between the expression level of the biomarkers and the clinical results observed.

Studies have shown a link between the presence of citrullinated anti-peptide antibodies at the basal level with a better response to an ABA treatment (Gottenberg et al., 2012). However, these studies did not use the strategy of combination of biomarkers to improve specificity or sensitivity and focus on RA patients naïve of all biotherapies. Studies on ABA relating to patients in rotation situation (with insufficient or inadequate response to at least one biotherapy) are mainly concentrated on the effectiveness of ABA and its therapeutic relevance compared to other bDMARDs (Harrold et al., 2015). One study reports that the phenotype of B and T cells is a factor to take into account in estimating the response to bDMARDs notably to that of ABA within a mixed population of naïve patients or in situation of rotation (Salomon et al., 2017). To the knowledge of the inventors, there is no data in the literature concerning the characterisation of biomarkers predictive of the response to ABA in patients having had an inadequate response to one or more treatment(s) with biotherapy.

There thus exists a need to identify novel methods and/or biomarkers making it possible to guide the practitioner in a personalised manner towards the treatment that is the most promising in terms of effectiveness for a given patient with a chronic inflammatory disease, in particular for patients with rheumatoid arthritis, and notably for those who are in a situation of inadequate response to a first treatment with biotherapy.

The present invention responds to this technical problem vis-à-vis the response to a treatment with a T-lymphocyte cell co-stimulation modulator agent for a patient with rheumatoid arthritis not having had a sufficient therapeutic response to one or more prior treatment(s) with biotherapy; the inventors having identified a set of biological biomarkers of which the expression level detected in a biological sample taken from such a patient makes it possible to estimate the probability of an effective response to said treatment in said patient.

### SUMMARY OF THE INVENTION

The present invention relates to a method for estimating the effectiveness of treatment with a T-lymphocyte cell co-stimulation modulator agent in a patient with rheumatoid arthritis and not having had an appropriate therapeutic response to one or more prior treatment(s) with biotherapy, said method comprising:

a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b) the estimation of said effectiveness of treatment with a T-lymphocyte cell co-stimulation modulator agent in said patient as a function of each expression level measured for a biomarker selected from said group.

In particular, the method for estimating the effectiveness of treatment with a T-lymphocyte cell co-stimulation modulator agent according to the invention comprises:

a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b1) the comparison of the expression level measured at step a) compared to that measured in a plurality of samples of patients with rheumatoid arthritis and having received treatment with a T-lymphocyte cell co-stimulation modulator agent for which the effectiveness of treatment is known; said comparison being carried out by means of a statistical learning model using the expression levels of at least two of the biomarkers measured at step a) as input data,
b2) the estimation of said effectiveness of treatment with a T-lymphocyte cell co-stimulation modulator agent in said patient as a function of the results determined by the model defined at step b1).

The set of biomarkers identified by the inventors is thus particularly suited to estimating the effectiveness of treatment with a T-lymphocyte cell co-stimulation modulator agent in a patient with rheumatoid arthritis.

The present invention furthermore relates to a system for estimating the effectiveness of treatment with a T-lymphocyte cell co-stimulation modulator agent in a patient with rheumatoid arthritis and who has had an inadequate response to at least one prior treatment with biotherapy, said system comprising:

means for measuring or receiving measurement data of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
means for processing measurement data configured to estimate said effectiveness of treatment in said patient as a function of each expression level measured for a biomarker selected from this group.

Preferably, the estimation method and the estimation system according to the invention make it possible to estimate the response to a T-lymphocyte cell co-stimulation modulator agent, in particular by blocking, directly or indirectly, the activation of the receptor CD28, in particular by preventing the fixation of the molecules CD80 or CD86 to the receptor CD28. Advantageously, the estimation method and the estimation system according to the invention make it possible to estimate the response to an agent that fixes itself to the molecules CD80/CD86, and in particular to estimate the response to Abatacept.

### BRIEF DESCRIPTION OF THE FIGURES

FIG. 1 represents the ROC (Receiver Operating Characteristic) curve obtained during the evaluation of the performances of the model with the 3 variables COMP, LBP and FN. It represents an example of the sensitivity of the test (Y-axis) as a function of the complementarity of the specificity of the test: 1—specificity (X-axis).

### DETAILED DESCRIPTION OF THE INVENTION

The problem encountered in the field of the invention for the development of a robust predictive test firstly consists in identifying the biomarkers which, taken together, make it possible to obtain a relevant prediction with both high specificity and high sensitivity.

That is why, according to a first aspect, the present invention relates to a method for estimating the effectiveness of treatment with a T-lymphocyte cell co-stimulation modulator agent in a patient with rheumatoid arthritis and who has had an inadequate response to at least one prior treatment with biotherapy, said method comprising, or even consisting in:

a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b) the estimation of said effectiveness of treatment with a T-lymphocyte cell co-stimulation modulator agent in said patient as a function of each expression level measured for a biomarker selected from said group.

The inventors have in fact identified sets or combinations of relevant biomarkers to estimate the effectiveness of treatment with a T-lymphocyte cell co-stimulation modulator agent in a patient with rheumatoid arthritis and who has had an inadequate response to at least one prior treatment with biotherapy, namely at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP).

In particular, the method for estimating the effectiveness of treatment with a T-lymphocyte cell co-stimulation modulator agent according to the invention comprises:

a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b1) the comparison of the expression level measured at step a) compared to that measured in a plurality of samples of patients with rheumatoid arthritis and having received treatment with a T-lymphocyte cell co-stimulation modulator agent for which the effectiveness of treatment is known; said comparison being carried out by means of a statistical learning model using the expression levels of at least two of the biomarkers measured at step a) as input data,
b2) the estimation of said effectiveness of treatment with a T-lymphocyte cell co-stimulation modulator agent in said patient as a function of the results determined by the model defined at step b1).

The measurement of the expression level of the particular combinations of these particular biomarkers and their analysis notably by means of a statistical learning model makes it possible to obtain a relevant estimation of the prediction of response to a T-lymphocyte cell co-stimulation modulator agent for a patient with rheumatoid arthritis.

With regard to step a) of the method according to the invention, the relevant biomarkers identified by the inventors are defined hereafter.

C4b complement binding protein (C4BP) is a protein involved in the complement system where it acts as an inhibitor. It inhibits the action of conventional pathways and lectins, more particularly C4. It also has the capacity of binding C3b. Excessive or poorly oriented activation of the complement contributes to the pathogenesis of inflammatory diseases such as rheumatoid arthritis (Swaak et al., 1987). The use of the recombinant protein C4BP in two murine RA models reduces the severity of the disease (Blom et al., 2009).

CRP (C-Reactive Protein), alpha 1 antitrypsin and the complement C4 are markers of the inflammatory status, the clinical use of which is very widespread and notably in the monitoring of chronic inflammatory rheumatisms (Buisseret et al., 1977; Vogt et al., 2017). These proteins belong to a category of proteins of which the expression strongly increases in acute phase. These cytokines, in their turn, signal stromal cells for the synthesis of secondary inflammatory mediators such as interleukin 6 (IL-6), interleukin 8 (IL-8) or instead monocytic chemical-attracting proteins. CRP is traditionally used to estimate the score of the activity of RA (Wells et al., 2009). The expression level of the complement C4 seems lower in RA patients who respond to Rituximab at 12 months (Conigliaro et al., 2016).

The protein COMP (Cartilage Oligomeric Matrix Protein), also designated Thrombospondin 5 (TSP 5), is a glycoprotein, a member of the family of extracellular proteins of thrombospondin. COMP is a calcium binding protein mainly present in the joint, nasal and tracheal cartilage. But its expression may be more widespread in other tissue types, including the synovia and the tendon. Intact COMP is homopentameric and binds to type I, II and IX collagens. It seems to have a structural role in endochondral ossification and in the assembly and stabilisation of the extracellular matrix by its interaction with collagen fibres and matrix components such as aggrecans. Moreover, COMP may also have a function of storage and distribution of hydrophobic cellular signalling molecules such as for example vitamin D. Mutations of the COMP gene lead to pseudo achondroplasia and certain forms of multiple epiphyseal dysplasia. COMP is considered as a marker of the degradation of the cartilage at the serum level in joint diseases and in particular in rheumatoid arthritis (Momohara et al., 2004). The expression level of COMP is higher in patients with aggressive RA in comparison with patients with less severe RA (El Defrawy et al., 2016). COMP has even been proposed as a biomarker comparable to the standard biomarkers usually used to estimate the activity of the disease such as CRP for example (Sakthiswary et al., 2017). For Crnkic and his colleagues, the serum concentration of COMP decreases at 3 months of treatment with Infliximab or Etanercept and remains low at 6 months in responders and non-responders (Crnkic et al., 2003). Conversely, for Kawashiri's team, the decrease in COMP is characteristic of responders at 6 months with a treatment with Etanercept (Kawashiri et al., 2010). Concerning the aspect of the prediction of response to bDMARDs, one study shows that the basal level of COMP in RA patients makes it possible to predict the effectiveness of treatment with Adalimumab, a TNF-alpha inhibitor (Morozzi et al., 2007).

Fibronectin (FN) is an omnipresent extracellular glycoprotein which exists in soluble form in body fluids and in insoluble form in the extracellular matrix (Maurer et al., 2015). In general, fibronectin is synthesised and present around the fibroblasts, endothelial cells, chondrocytes, glial cells and myocytes. Extremely high levels of glycoprotein are found in the plasma. Fibronectin is a ligand for numerous molecules, including fibrin, heparin, chondroitin sulphate, collagen/gelatine, various integrins and growth factors, myocilin and apolipoprotein A. It is involved in multiple cellular processes such as cell adhesion/migration, blood coagulation, morphogenesis, tissue repair, embryogenesis and cell signalling. The involvement of fibronectin in rheumatoid arthritis has been very little described. The molecular status of fibronectin makes it possible to distinguish RA patients from healthy patients (Cheng et al., 2014) or from patients with other pathologies such as lupus erythematosus (Przybysz et al., 2013). The specific presence of citrullinated fibronectin and citrullinated anti-fibronectin antibody has been described in the synovia of RA patients (Kimura et al., 2014). To the knowledge of the inventors, the predictive aspect of fibronectin as biomarker in the treatment of RA with bDMARDs has not been reported in the literature.

Lipopolysaccharide Binding Protein (LBP) is an acute phase protein which binds to various LPS molecules and to lipid A (Schumann et al., 1990). LBP is constitutively produced by hepatocytes in the liver. It binds to LPS (lipopolysaccharide) and presents it to the receptor CD14 present on monocytic cells. In the presence of LBP, cytokines are released by monocytes at lower LPS concentrations compared to those produced in the absence of LBP. Thus, the main function of LBP is to improve the capacity of the host to detect LPS at the start of the infection. One of the LBP functions leads to LPS neutralisation. In addition, LBP associates itself with the lipid portion A of gram-negative bacteria leading to its opsonisation. In normal serum, LBP is constitutively present and its concentration can increase by 10 times in acute phase response (Prucha et al., 2003). Few studies recount the relation between LBP and rheumatoid arthritis. Two studies suggest however that LBP is a marker of inflammation in RA patients (Heumann et al., 1995) and may represent a new marker of the activity of the disease in RA (Wen et al., 2017).

Preferably, the method for estimating the effectiveness of treatment according to the invention is based on the in vitro measurement of the expression level of at least three of the five aforementioned biomarkers, of at least four or of the five biomarkers of the aforementioned group, namely C4b-Binding Protein (C4BP), C-Reactive Protein (CRP), Cartilage Oligomeric Matrix Protein (COMP), Fibronectin (FN) and Lipopolysaccharide-Binding Protein (LBP).

The preferred embodiments of the method for estimating the effectiveness of treatment according to the invention comprise the in vitro measurement of the expression level of the particular combinations of the following biomarkers among the list of the five aforementioned biomarkers:
at least COMP and FN or at least COMP and CRP, more preferably at least COMP and FN;
at least COMP, LBP and CRP or at least COMP, CRP and C4BP, or further and more preferably at least COMP, LBP and FN or at least COMP, LBP and C4BP.

These sets make it possible to obtain the most relevant results in terms of estimating treatment effectiveness.

The method according to the invention makes it possible to estimate the effectiveness of treatment with a T-lymphocyte cell co-stimulation modulator agent in a patient with rheumatoid arthritis who has had an inadequate response to at least one prior treatment with a biotherapy.

“Patient with rheumatoid arthritis who has had an inadequate response” is taken to mean a patient with rheumatoid arthritis who has had an insufficient response to at least one prior treatment with a biotherapy, but also a patient with rheumatoid arthritis who has had a satisfactory response to at least one prior treatment with a biotherapy but who has presented at least one adverse event of moderate to severe intensity during the prior treatment(s) requiring the stoppage of treatment.

“Insufficient response to at least one prior treatment with a biotherapy” is taken to mean to designate a patient not having presented a positive therapeutic response to one or more prior treatment(s) with a bDMARD (biological Disease-Modifying Anti-Rheumatic Drug). Within the context of the treatment of chronic inflammatory diseases, current therapeutic strategies are conducted to reduce the activity of the rheumatism and the response to the treatment is generally assessed at 6 months. Concerning rheumatoid arthritis, the response to treatment is determined by the evolution of the activity of the rheumatism according to the EULAR (European League Against Rheumatism) response. The EULAR response takes into account the activity of the rheumatism which is evaluated by the DAS28 (Disease Activity Score 28) as well as its variation. The DAS is a composite score calculated on the basis of the number of painful joints out of 28 joints, VAS (Visual Analogue Scale), and a biological inflammatory parameter: SR (Sedimentation Rate) or CRP, as described for example by Wells et al., 2009 and Fransen and van Riel, 2005. The EULAR response is defined as a function of the DAS28 score at time T and the difference between the DAS28 at time T and the initial DAS28, that is to say before treatment. In the context of the present invention, “insufficient response to a treatment” is taken to mean in particular a EULAR response with a DAS28 at 6 months greater than 3.2 or a variation in DAS28 at 6 months and a DAS28 before treatment less than or equal to 1.2.

In other words, “an insufficient response” may also be defined by a variation in DAS28 at 6 months and DAS28 before treatment less than or equal to 0.6. Conversely, according to the invention “a sufficient response” is defined by a DAS28 at 6 months less than or equal to 3.2 associated with a variation in DAS28 at 6 months and DAS28 before treatment greater than 1.2. Consequently, according to the invention, a so-called moderate response according to the EULAR response is considered as an insufficient response.

Biotherapy is taken to mean a therapy resorting to the use of a bDMARD. DMARDs are a category of drugs defined by their use in rheumatoid arthritis to slow down the progression of the disease. Several types of DMARDs exist, classed in the following manner:
synthetic DMARDs (sDMARDs) which comprise conventional synthetics (csDMARDs) and targeted synthetics (tsDMARDs). csDMARDs are traditional drugs such as methotrexate, sulfasalazine, leflunomide, hydroxychloroquine, gold salts, etc. tsDMARDs are drugs which have been developed to target a particular molecular structure.
biological DMARDs (bDMARDs) which comprise original biological DMARDs (boDMARDs) and biosimilar DMARDs (bsDMARDs). bsDMARDs are those which have the same primary, secondary and tertiary structure as the original biological treatment (boDMARD) and have an effectiveness and a safety similar to those of the original protein.

“Sufficient response to a treatment” is taken to mean designating a patient having presented a sufficient therapeutic response to one or more prior treatment(s) with a bDMARD (biological Disease-Modifying Anti-Rheumatic Drug). In the context of the present invention, “sufficient response to a treatment” is taken to mean in particular a EULAR response with a DAS28 at 6 months less than or equal to 3.2 associated with a variation in DAS28 at 6 months and DAS28 before treatment greater than 1.2.

“Adverse event”, or AE, is taken to mean any untoward occurrence in a patient, whether this occurrence is linked or not to the treatment with biotherapy. If this adverse event is considered by the physician as having a scientifically reasonable causality link with the procedure, the method, the act or the treatment, it is qualified as an adverse effect. The expression “scientifically reasonable causality link” signifies that there exists proof or an argument making it possible to suggest, in scientific terms, a cause and effect relationship between the untoward and undesired reaction observed and the procedure, the method, the act or the treatment.

The intensity of adverse events is evaluated by the physician with the aid of the following classification, well known in the field:
grade 1 mild intensity: adverse event generally transitional and not interfering with everyday activities.
grade 2 moderate intensity: adverse event sufficiently discomforting to interfere with everyday activities.
grade 3 severe intensity: adverse event considerably modifying the normal course of activities of the subject, or invalidating, or constituting a threat to the life of a subject.

The grades of all known adverse events as a function of pathologies are listed by the National Cancer Institute and accessible on the web site of the National Institutes of Health (Common Terminology Criteria for Adverse Events (CTCAE); https://safetyprofiler-ctep.nci.nih.gov/CTC/CTC.aspx).

The different adverse events linked to a treatment with biotherapy are notably classed in the Summary of Product Characteristics (SmPC).

Preferably, the method according to the invention makes it possible to estimate the effectiveness of treatment with a T-lymphocyte cell co-stimulation modulator agent in a patient with rheumatoid arthritis and who has had an inadequate response to at least one prior treatment with an anti-TNFa biotherapy. Even more preferably, the method according to the invention makes it possible to estimate the effectiveness of treatment with a T-lymphocyte cell co-stimulation modulator agent in a patient with rheumatoid arthritis who has had an inadequate response to at least one prior treatment selected from Etanercept, Adalimumab, Infliximab, Tocilizumab, Rituximab, Certolizumab and Golimumab, and preferably only one of these treatments.

According to the invention, the method makes it possible to estimate the effectiveness of treatment with a T-lymphocyte cell co-stimulation modulator agent. Such an agent may be defined as being an agent that is capable of blocking, or even inhibiting, directly or indirectly, T-lymphocyte cell co-stimulation, in particular by blocking or inhibiting, directly or indirectly, the co-stimulatory pathway of these cells by inactivation of the receptor CD28, notably by preventing the fixation of the molecules CD80 or CD86 to the receptor CD28. Among these agents, the bDMARD Abatacept may notably be cited.

In a particularly advantageous manner, the method according to the invention makes it possible to estimate the effectiveness of treatment with a T-lymphocyte cell co-stimulation modulator agent which is Abatacept.

Any biological sample constituted of a biological fluid may be used in the context of the invention for measuring in vitro the expression level of the biomarkers and combinations of biomarkers mentioned above, and among which may notably be cited synovial liquid, serum, plasma, saliva, urine, etc, preferably serum.

According to a preferred embodiment, the expression level of the biomarkers and combinations of biomarkers mentioned above is measured in vitro on a sample of serum from the patient for whom it is sought to estimate the effectiveness of treatment with a T-lymphocyte cell co-stimulation modulator agent.

Advantageously, the method for estimating the effectiveness of treatment according to the invention comprises at step a) the in vitro measurement of the expression level of the protein biomarkers or combinations of protein biomarkers.

Particularly preferred embodiments of the method according to the invention are the following, each being to apply to the combinations of biomarkers defined previously, namely at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP):
the estimation of the effectiveness of treatment with Abatacept, in a patient with rheumatoid arthritis;
the estimation of the effectiveness of treatment with Abatacept, in a patient with rheumatoid arthritis comprising the measurement of the protein expression level;
the estimation of the effectiveness of treatment with Abatacept, in a patient with rheumatoid arthritis and who has had an inadequate response to at least one prior treatment with a biotherapy selected from Etanercept, Adalimumab, Infliximab, Tocilizumab, Rituximab, Certolizumab and Golimumab, preferably to only one of these treatments;
the estimation of the effectiveness of treatment with Abatacept, in a patient with rheumatoid arthritis and who has had an inadequate response to at least one prior treatment with a biotherapy selected from Etanercept, Adalimumab Tocilizumab, Infliximab, Rituximab, Certolizumab and Golimumab, comprising the measurement of the protein expression level, preferably to only one of these treatments;
the estimation of the effectiveness of treatment with Abatacept, in a patient with rheumatoid arthritis and who has had an insufficient response to at least one prior treatment with a biotherapy selected from Etanercept, Adalimumab, Infliximab, Tocilizumab, Rituximab, Certolizumab and Golimumab, preferably to only one of these treatments;
the estimation of the effectiveness of treatment with Abatacept, in a patient with rheumatoid arthritis and who has had an insufficient response to at least one prior treatment with a biotherapy selected from Etanercept, Adalimumab Tocilizumab, Infliximab, Rituximab, Certolizumab and Golimumab, comprising the measurement of the protein expression level, preferably to only one of these treatments.

At step b1) of the estimation method according to the invention, the expression level of the biomarkers or combinations of biomarkers measured at step a) described above is compared to that measured in a plurality of samples of patients with rheumatoid arthritis and having received a treatment with a T-lymphocyte cell co-stimulation modulator agent for which the effectiveness of treatment is known.

This comparison is carried out by means of a statistical learning model using the expression levels of at least two of the biomarkers measured at step a) as input data. To do so, any statistical learning model may be used, and notably the models obtained by logistic regression methods, discriminant analysis, neural networks, decision tree learning, support vector machines (SVM), or aggregation of models.

Preferably, in the method for estimating the effectiveness of treatment with a T-lymphocyte cell co-stimulation modulator agent according to the invention, the expression levels of each biomarker measured at step a) are used to obtain a score linked to the estimation of the effectiveness of treatment in said patient, said score being compared to at least one predetermined threshold so as to classify the prognosis among a plurality of classes. In this embodiment, it is notably possible to use a plurality of classes which comprises at least two classes of which one class of non-response to the treatment by said T-lymphocyte cell co-stimulation modulator agent, and preferably which comprises, or even consists in, two classes of which one class of non-response to the treatment with said T-lymphocyte cell co-stimulation modulator agent. The two classes may for example be derived from patients designated “non-responders”, in whom treatment with a T-lymphocyte cell co-stimulation modulator is ineffective and patients designated “responders” in whom treatment with a T-lymphocyte cell co-stimulation modulator is effective. Other classes may be envisaged to refine the analyses, in particular in addition to those of so-called “non-responder” patients, such as for example “moderate responders” and “good responders” to the treatment, the qualifications of moderate or good corresponding to the usual assessment of practitioners specialising in the treatment of rheumatoid arthritis. Still in this embodiment, the estimation of the effectiveness of treatment in the patient with rheumatoid arthritis and who has had an inadequate response to at least one prior treatment with biotherapy comprises the comparison of said score with a predetermined threshold below which poor effectiveness is predicted and above which good effectiveness is predicted.

Preferably, the method for estimating the effectiveness of treatment with a T-lymphocyte cell co-stimulation modulator agent according to the invention uses at step b1) a learning model based on a prior analysis of samples of a cohort comprising patients treated with a T-lymphocyte cell co-stimulation modulator agent presenting good responses to the treatment and patients treated with a T-lymphocyte cell co-stimulation modulator agent presenting poor responses to the treatment. In this embodiment, the learning model is preferably based on a prior analysis which comprises the application of a method for learning and for selecting variables. Advantageously, logistic regression will be used as method for learning and for selecting variables.

Still in this embodiment based on a prior analysis which comprises the application of a method for learning and for selecting variables, the expression levels of the biomarkers or combinations of biomarkers measured at step a) are weighted as a function of the prior analysis of the cohort comprising patients treated with a T-lymphocyte cell co-stimulation modulator agent presenting good responses to the treatment and patients treated with a T-lymphocyte cell co-stimulation modulator agent presenting poor responses to the treatment to derive the score linked to the estimation of the effectiveness of treatment.

Still in this embodiment based on a prior analysis which comprises the application of a method for learning and for selecting variables, the method for estimating the effectiveness of treatment according to the invention may use a method of learning by decision tree. According to this embodiment, the expression levels of the biomarkers or combinations of biomarkers measured at step a) are compared to a reference value at each node of the tree.

The reference values may be obtained by the analysis of the expression level of the biomarkers or combinations of biomarkers in biological samples of a set of patients with rheumatoid arthritis before treatment so as to have available a set of data on the expression levels of the biomarkers associated with each biological sample of each patient.

These reference values can change over time as a function of the results obtained with other patients that come and complete the number of results serving to define the threshold value.

According to a second aspect, the invention also relates to a system for estimating the effectiveness of treatment with a T-lymphocyte cell co-stimulation modulator agent in a patient with rheumatoid arthritis and who has had an inadequate response to at least one prior treatment with biotherapy, said system comprising:
means for measuring or receiving measurement data of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
means for processing measurement data configured to estimate said effectiveness of treatment in said patient as a function of each expression level measured for a biomarker selected from this group.

Among the means for measuring the expression level of the biomarkers or combinations of biomarkers selected, it is notably possible to cite specific reagents of each of the biomarkers such as enzymes, substrates or antibodies which may be used in methods, such as among others nephelometry, chemiluminescence, immunoturbidimetry, flow cytometry, ELISA, etc, but also physical means such as mass spectrometry analysis methods for example.

The system according to the invention may moreover contain means for receiving measurement data, thus making it possible to estimate, for said patient, the effectiveness of response to treatment by the T-lymphocyte cell co-stimulation modulator agent, from data supplied for example by a practitioner having had the expression level of the biological biomarkers measured such as described previously.

The reception means may notably comprise transmission/reception means for exchanging with a remote server through a communication network such as an intranet network or the secure internet network. The device may also comprise input means such as a keyboard.

The data processing means may notably call on database management, code instructions, the development of software comprising an algorithmic brick, an interface to enable the user to consult the results, etc. These different elements may be recorded on a storage support such as a hard disc, a CD ROM, a USB key, or any other storage support known to those skilled in the art.

They may be implemented by a device which may be fixed or mobile. The device is for example a personal computer, a mobile telephone, an electronic tablet, or any other type of terminal known to those skilled in the art.

In an alternative, the system may also comprise transmission means for transmitting, still via intranet or internet for example, the results of the estimation of the effectiveness of treatment in the patient concerned.

According to another advantageous alternative, the system according to the invention comprises means for receiving the effectiveness data obtained, and does so in order to complete and enrich the reference values in view of the result of treatment obtained with regard to the expression level of the selected biomarkers.

### EXAMPLES

### Materials & Methods

### Development of the Predictive Model:

The link between the response to the biotherapy and each variable is analysed through a logistic regression model on a set of data. The variable to explain is the good response at 6 months.

Firstly, a pre-selection of the variables to include in the multivariate model is carried out. To do so, the predictive capacity of each explanatory variable is analysed individually. The biomarkers are analysed in a quantitative and qualitative manner. A method for selecting variables is put in place to conserve uniquely the relevant variables that will next be introduced into a multivariate model. The biomarkers are pre-selected if they have:
In quantitative form, a p-value<0.25 or an AUC>0.60
In qualitative form, a p-value<0.05 and an AUC>0.65

The criteria chosen are voluntarily wide to include significant variables, but also variables presenting trends in the multivariate model.

The preselected biomarkers exhibit relevant trends to analyse. Multivariate models with the different possible combinations of these biomarkers are constructed, and the AUC calculated. Models having an AUC>0.75 are considered as relevant and are conserved.

The models thereby constructed make it possible to weight the dosage results of each of the specific biomarkers to obtain a probability of response. The coefficients of each model make it possible to calculate from the dosage values of each patient an associated probability of response. The performance characteristics (AUC (area under the curve), sensitivity and specificity, PPV (positive predictive value) and NPV (negative predictive value)) of each model are calculated to define its relevance.

An AUC>0.70 is considered as an acceptable discrimination, an AUC>0.80 demonstrates very good discrimination capacity. A level of probability threshold is fixed to calculate specificity and sensitivity. This optimal threshold is determined on the basis of the Youden index. At this threshold, the patients may be classified as a function of the following table 1:

TABLE 1

| | Responder | Non responder |
|---|---|---|
| Positive test (proba > threshold) | TP | FP |
| Negative test (proba < threshold) | FN | TN |

TP (true positives) represents the number of responder individuals with a positive test,
FP (false positives) represents the number of non-responder individuals with a positive test,
FN (false negatives) represents the number of responder individuals with a negative test,
TN (true negatives) represents the number of non-responder individuals with a negative test.

The sensitivity, or the probability that the test is positive if the patient is a responder, is measured in sufferers only. It is given by:

$$\frac{T P}{T P + F N} .$$

The specificity is measured in non-sufferers only. The specificity, or the probability of obtaining a negative test in non-responders is given by:

$$\frac{T N}{T N + F P}$$

The sensitivity of the test measures its capacity to give a positive result when the patient is a responder. The specificity measures the capacity of the test to give a negative result when the patient is a non-responder.

The positive predictive value (PPV) is the probability that the patient is a responder when the test is positive.

$$P P V = \frac{T P}{T P + F P} .$$

The negative predictive value (NPV) is the probability that the patient is not a responder when the test is negative.

$$N P V = \frac{T N}{T N + F N} .$$

### Results:

Out of our learning cohort (30 RA patients), the models constructed have the characteristics presented in table 2 below which presents combinations with AUC>0.75. All the combinations including at least 2 of the 5 biomarkers among C4BP, CRP, COMP, FN and LBP provide relevant results.

TABLE 2

| Number of | | | | | | |
|---|---|---|---|---|---|---|
| variables of the | | | | | | |
| model | COMP | LBP | CRP | C4BP | FN | AUC |
| 2 | x | | | | x | 0.88 |
| | x | | x | | | 0.88 |
| | x | x | | | | 0.86 |
| | x | | | x | | 0.85 |
| | | | x | x | | 0.83 |
| | | x | | x | | 0.81 |
| | | | | x | x | 0.80 |
| | | x | x | | | 0.80 |
| | | x | | | x | 0.76 |
| 3 | x | x | x | | | 0.96 |
| | x | | x | x | | 0.93 |
| | x | x | | | x | 0.91 |
| | x | x | | x | | 0.91 |
| | x | | | x | x | 0.90 |
| | x | | x | | x | 0.90 |
| | | x | x | x | | 0.89 |
| | | | x | x | x | 0.85 |
| | | x | | x | x | 0.85 |
| | | x | x | | x | 0.81 |

Taking for example the model with 3 variables COMP, LBP and FN, the characteristics obtained are presented in table 3 below:

TABLE 3
Model with 3 variables: COMP, LBP and FN

| | |
|---|---|
| AUC | 0.909 |
| Sensitivity | 1 |
| Specificity | 0.82 |
| PPV | 0.67 |
| NPV | 1 |

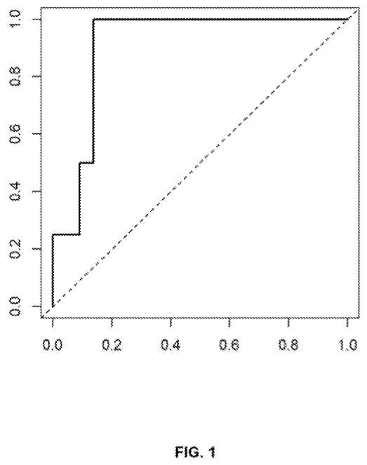

The corresponding ROC curve is represented in FIG. 1.

### REFERENCES

Blom, A. M., Nandakumar, K. S., and Holmdahl, R. (2009). C4b-binding protein (C4BP) inhibits development of experimental arthritis in mice. Ann. Rheum. Dis. 68, 136-142.
Briggs, M. D., Wright, M. J., and Mortier, G. R. (1993). Multiple Epiphyseal Dysplasia, Autosomal Dominant. In GeneReviews(®), M. P. Adam, H. H. Ardinger, R. A. Pagon, S. E. Wallace, L. J. Bean, H. C. Mefford, K. Stephens, A. Amemiya, and N. Ledbetter, eds. (Seattle (Wash.): University of Washington, Seattle), p.
Buisseret, P. D., Pembrey, M. E., and Lessof, M. H. (1977). alpha 1-antitrypsin phenotypes in rheumatoid arthritis and ankylosing spondylitis. Lancet Lond. Engl. 2, 1358-1359.
Chen, F. H., Herndon, M. E., Patel, N., Hecht, J. T., Tuan, R. S., and Lawler, J. (2007). Interaction of cartilage oligomeric matrix protein/thrombospondin 5 with aggrecan. J. Biol. Chem. 282, 24591-24598.
Cheng, Y., Chen, Y., Sun, X., Li, Y., Huang, C., Deng, H., and Li, Z. (2014). Identification of potential serum biomarkers for rheumatoid arthritis by high-resolution quantitative proteomic analysis. Inflammation 37, 1459-1467.
Conigliaro, P., Triggianese, P., Chimenti, M. S., Lucchetti, R., Kroegler, B., and Perricone, R. (2016). Serological markers associated with disease activity in patients with rheumatoid arthritis treated with rituximab. J. Int. Med. Res. 44, 53-57.
Crnkic, M., Månsson, B., Larsson, L., Geborek, P., Heinegård, D., and Saxne, T. (2003). Serum cartilage oligomeric matrix protein (COMP) decreases in rheumatoid arthritis patients treated with infliximab or etanercept. Arthritis Res. Ther. 5, R181-185.
Derambure, C., Dzangue-Tchoupou, G., Berard, C., Vergne, N., Hiron, M., D'Agostino, M. A., Musette, P., Vittecoq, O., and Lequerré, T. (2017). Pre-silencing of genes involved in the electron transport chain (ETC) pathway is associated with responsiveness to abatacept in rheumatoid arthritis. Arthritis Res. Ther. 19, 109.
El Defrawy, A. O., Gheita, T. A., Raslan, H. M., El Ansary, M. M., and El Awar, A. H. (2016). Serum and synovial cartilage oligomeric matrix protein levels in early and established rheumatoid arthritis. Z. Rheumatol. 75, 917-923.
Fransen, J., and van Riel, P. L. C. M. (2005). The Disease Activity Score and the EULAR response criteria. Clin. Exp. Rheumatol. 23, S93-99.
Gottenberg, J. E., Ravaud, P., Cantagrel, A., Combe, B., Flipo, R. M., Schaeverbeke, T., Houvenagel, E., Gaudin, P., Loeuille, D., Rist, S., et al. (2012). Positivity for anti-cyclic citrullinated peptide is associated with a better response to abatacept: data from the “Orencia and Rheumatoid Arthritis” registry. Ann. Rheum. Dis. 71, 1815-1819.
Harrold, L. R., Reed, G. W., Kremer, J. M., Curtis, J. R., Solomon, D. H., Hochberg, M. C., and Greenberg, J. D. (2015). The comparative effectiveness of abatacept versus anti-tumour necrosis factor switching for rheumatoid arthritis patients previously treated with an anti-tumour necrosis factor. Ann. Rheum. Dis. 74, 430-436.
Heumann, D., Bas, S., Gallay, P., Le Roy, D., Barras, C., Mensi, N., Glauser, M. P., and Vischer, T. (1995). Lipopolysaccharide binding protein as a marker of inflammation in synovial fluid of patients with arthritis: correlation with interleukin 6 and C-reactive protein. J. Rheumatol. 22, 1224-1229.
Kawashiri, S.-Y., Kawakami, A., Ueki, Y., Imazato, T., Iwamoto, N., Fujikawa, K., Aramaki, T., Tamai, M., Nakamura, H., Origuchi, T., et al. (2010). Decrement of serum cartilage oligomeric matrix protein (COMP) in rheumatoid arthritis (RA) patients achieving remission after 6 months of etanercept treatment: comparison with CRP, IgM-RF, MMP-3 and anti-CCP Ab. Jt. Bone Spine Rev. Rhum. 77, 418-420.
Kimura, E., Kanzaki, T., Tahara, K., Hayashi, H., Hashimoto, S., Suzuki, A., Yamada, R., Yamamoto, K., and Sawada, T. (2014). Identification of citrullinated cellular fibronectin in synovial fluid from patients with rheumatoid arthritis. Mod. Rheumatol. 24, 766-769.
Krintel, S. B., Dehlendorff, C., Hetland, M. L., Horsley-Petersen, K., Andersen, K. K., Junker, P., Pødenphant, J., Ellingsen, T., Ahlquist, P., Lindegaard, H. M., et al. (2016). Prediction of treatment response to adalimumab: a double-blind placebo-controlled study of circulating microRNA in patients with early rheumatoid arthritis. Pharmacogenomics J. 16, 141-146.
Luo, H., Yu, S., Lin, Y., Guo, Q., Ma, R., Ye, Z., Di, Y., Li, N., Miao, Y., Zhou, Y., et al. (2016). A novel deleterious mutation in the COMP gene that causes pseudoachondroplasia. Hum. Genome Var. 3, 16009.
Maurer, L. M., Ma, W., and Mosher, D. F. (2015). Dynamic structure of plasma fibronectin. Crit. Rev. Biochem. Mol. Biol. 51, 213-227.
Momohara, S., Yamanaka, H., Holledge, M. M., Mizumura, T., Ikari, K., Okada, N., Kamatani, N., and Tomatsu, T. (2004). Cartilage oligomeric matrix protein in serum and synovial fluid of rheumatoid arthritis: potential use as a marker for joint cartilage damage. Mod. Rheumatol. 14, 356-360.
Morozzi, G., Fabbroni, M., Bellisai, F., Cucini, S., Simpatico, A., and Galeazzi, M. (2007). Low serum level of COMP, a cartilage turnover marker, predicts rapid and high ACR70 response to adalimumab therapy in rheumatoid arthritis. Clin. Rheumatol. 26, 1335-1338.
Nakamura, S., Suzuki, K., Iijima, H., Hata, Y., Lim, C. R., Ishizawa, Y., Kameda, H., Amano, K., Matsubara, K., Matoba, R., et al. (2016). Identification of baseline gene expression signatures predicting therapeutic responses to three biologic agents in rheumatoid arthritis: a retrospective observational study. Arthritis Res. Ther. 18, 159.
Prajapati, R., Plant, D., and Barton, A. (2011). Genetic and genomic predictors of anti-TNF response. Pharmacogenomics 12, 1571-1585.
Prucha, M., Herold, I., Zazula, R., Dubska, L., Dostal, M., Hildebrand, T., and Hyanek, J. (2003). Significance of lipopolysaccharide-binding protein (an acute phase protein) in monitoring critically ill patients. Crit. Care Lond. Engl. 7, R154-159.
Przybysz, M., Borysewicz, K., and Kqtnik-Prastowska, I. (2013). Fibronectin molecular status determination useful to differentiate between rheumatoid arthritis and systemic lupus erythematosus patients. Rheumatol. Int. 33, 37-43.
Rendas-Baum, R., Wallenstein, G. V., Koncz, T., Kosinski, M., Yang, M., Bradley, J., and Zwillich, S. H. (2011). Evaluating the efficacy of sequential biologic therapies for rheumatoid arthritis patients with an inadequate response to tumor necrosis factor-α inhibitors. Arthritis Res. Ther. 13, R25.
Sakthiswary, R., Rajalingam, S., Hussein, H., Sridharan, R., and Asrul, A. W. (2017). Cartilage oligomeric matrix protein (COMP) in rheumatoid arthritis and its correlation with sonographic knee cartilage thickness and disease activity. Clin. Rheumatol.
Salomon, S., Guignant, C., Morel, P., Flahaut, G., Brault, C., Gourguechon, C., Fardellone, P., Marolleau, J.-P., Gubler, B., and Goëb, V. (2017). Th17 and CD24(hi)CD27(+) regulatory B lymphocytes are biomarkers of response to biologics in rheumatoid arthritis. Arthritis Res. Ther. 19, 33.
Scarsi, M., Ziglioli, T., and Airo', P. (2011). Baseline numbers of circulating CD28-negative T cells may predict clinical response to abatacept in patients with rheumatoid arthritis. J. Rheumatol. 38, 2105-2111.
Schumann, R. R., Leong, S. R., Flaggs, G. W., Gray, P. W., Wright, S. D., Mathison, J. C., Tobias, P. S., and Ulevitch, R. J. (1990). Structure and function of lipopolysaccharide binding protein. Science 249, 1429-1431.
Smith, S. L., Plant, D., Eyre, S., and Barton, A. (2013). The potential use of expression profiling: implications for predicting treatment response in rheumatoid arthritis. Ann. Rheum. Dis. 72, 1118-1124.
Smolen, J. S., Landewé, R., Bijlsma, J., Burmester, G., Chatzidionysiou, K., Dougados, M., Nam, J., Ramiro, S., Voshaar, M., van Vollenhoven, R., et al. (2017). EULAR recommendations for the management of rheumatoid arthritis with synthetic and biological disease-modifying antirheumatic drugs: 2016 update. Ann. Rheum. Dis. 76, 960-977.
Souto, A., Maneiro, J. R., and Gómez-Reino, J. J. (2016). Rate of discontinuation and drug survival of biologic therapies in rheumatoid arthritis: a systematic review and meta-analysis of drug registries and health care databases. Rheumatol. Oxf. Engl. 55, 523-534.
Swaak, A. J., Van Rooyen, A., Planten, O., Han, H., Hattink, O., and Hack, E. (1987). An analysis of the levels of complement components in the synovial fluid in rheumatic diseases. Clin. Rheumatol. 6, 350-357.
Tatar, Z., Migne, C., Petera, M., Gaudin, P., Lequerre, T., Marotte, H., Tebib, J., Pujos Guillot, E., and Soubrier, M. (2016). Variations in the metabolome in response to disease activity of rheumatoid arthritis. BMC Musculoskelet. Disord. 17, 353.
Trocmé, C., Marotte, H., Baillet, A., Pallot-Prades, B., Garin, J., Grange, L., Miossec, P., Tebib, J., Berger, F., Nissen, M. J., et al. (2009). Apolipoprotein A-I and platelet factor 4 are biomarkers for infliximab response in rheumatoid arthritis. Ann. Rheum. Dis. 68, 1328-1333.
Vogt, L. M., Talens, S., Kwasniewicz, E., Scavenius, C., Struglics, A., Enghild, J. J., Saxne, T., and Blom, A. M. (2017). Activation of Complement by Pigment Epithelium-Derived Factor in Rheumatoid Arthritis. J. Immunol. Baltim. Md 1950 199, 1113-1121.
Wells, G., Becker, J.-C., Teng, J., Dougados, M., Schiff, M., Smolen, J., Aletaha, D., and van Riel, P. L. C. M. (2009). Validation of the 28-joint Disease Activity Score (DAS28) and European League Against Rheumatism response criteria based on C-reactive protein against disease progression in patients with rheumatoid arthritis, and comparison with the DAS28 based on erythrocyte sedimentation rate. Ann. Rheum. Dis. 68, 954-960.
Wen, W., Li, Y., Cheng, Y., He, J., Jia, R., Li, C., Guo, J., Sun, X., and Li, Z. (2017). Lipopolysaccharide-binding protein is a sensitive disease activity biomarker for rheumatoid arthritis. Clin. Exp. Rheumatol.
Zhang, J., Shan, Y., Reed, G., Kremer, J., Greenberg, J. D., Baumgartner, S., and Curtis, J. R. (2011). Thresholds in disease activity for switching biologics in rheumatoid arthritis patients: experience from a large U.S. cohort. Arthritis Care Res. 63, 1672-1679.

1. Method for estimating the effectiveness of treatment with a T-lymphocyte cell co-stimulation modulator agent in a patient with rheumatoid arthritis and who has had an inadequate response to at least one prior treatment with biotherapy, said method comprising:
a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b) the estimation of said effectiveness of treatment with a T-lymphocyte cell co-stimulation modulator agent in said patient as a function of each expression level measured for a biomarker selected from said group.

2. Method for estimating the effectiveness of treatment with a T-lymphocyte cell co-stimulation modulator agent according to claim 1, said method comprising:
a) the in vitro measurement of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
b1) the comparison of the expression level measured at step a) compared to that measured in a plurality of samples of patients with rheumatoid arthritis and having received treatment with a T-lymphocyte cell co-stimulation modulator agent for which the effectiveness of treatment is known; said comparison being carried out by means of a statistical learning model using the expression levels of at least two of the biomarkers measured at step a) as input data,
b2) the estimation of said effectiveness of treatment with a T-lymphocyte cell co-stimulation modulator agent in said patient as a function of the results determined by the model defined at step b1).

3. Method for estimating the effectiveness of treatment with a T-lymphocyte cell co-stimulation modulator agent according to any of claim 1 or 2, characterised in that the expression levels of each biomarker measured at step a) are used to obtain a score linked to the estimation of the effectiveness of treatment in said patient, said score being compared to at least one predetermined threshold so as to classify the prognosis among a plurality of classes.

4. Method for estimating the effectiveness of treatment with a T-lymphocyte cell co-stimulation modulator agent according to claim 3, characterised in that said plurality of classes comprises at least two classes of which one class of non-response to the treatment with said T-lymphocyte cell co-stimulation modulator agent.

5. Method for estimating the effectiveness of treatment with a T-lymphocyte cell co-stimulation modulator agent according to any of claims 3 to 4, characterised in that the estimation of said effectiveness of treatment in said patient comprises the comparison of said score with a predetermined threshold below which poor effectiveness is predicted and above which good effectiveness is predicted.

6. Method for estimating the effectiveness of treatment with a T-lymphocyte cell co-stimulation modulator agent according to any of claims 2 to 5, wherein the learning model is based on a prior analysis of samples of a cohort comprising patients treated with a T-lymphocyte cell co-stimulation modulator agent presenting good responses to the treatment and patients treated with a T-lymphocyte cell co-stimulation modulator agent presenting poor responses to the treatment.

7. Method for estimating the effectiveness of treatment with a T-lymphocyte cell co-stimulation modulator agent according to claim 6, wherein said prior analysis comprises the application of a method for learning and for selecting variables.

8. Method for estimating the effectiveness of treatment with a T-lymphocyte cell co-stimulation modulator agent according to claim 7, wherein said method for learning and for selecting variables is logistic regression.

9. Method for estimating the effectiveness of treatment with a T-lymphocyte cell co-stimulation modulator agent according to any of claims 6 to 8, wherein said expression levels are weighted as a function of the prior analysis of said cohort to derive said score.

10. Method for estimating the effectiveness of treatment with a T-lymphocyte cell co-stimulation modulator agent according to any of claims 7 to 9, wherein said learning method comprises a decision tree wherein each node corresponds to a comparison of the expression level measured at step a) with a reference value.

11. Method for estimating the effectiveness of treatment with a T-lymphocyte cell co-stimulation modulator agent according to any of the preceding claims, characterised in that said agentis capable of blocking or inhibiting, directly or indirectly, the co-stimulatory pathway of T-lymphocyte cells, by inactivation of the receptor CD28, and preferably said agent is Abatacept.

12. Method for estimating the effectiveness of treatment with a T-lymphocyte cell co-stimulation modulator agent according to any of the preceding claims, characterised in that said patient has had an inadequate response to at least one prior treatment selected from Etanercept, Adalimumab, Infliximab, Tocilizumab, Rituximab, Certolizumab, and Golimumab.

13. Method for estimating the effectiveness of treatment with a T-lymphocyte cell co-stimulation modulator agent according to any of the preceding claims, characterised in that the biological sample is constituted of a sample of biological fluid, and preferably serum.

14. Method for estimating the effectiveness of treatment with a T-lymphocyte cell co-stimulation modulator agent according to any of the preceding claims, characterised in that the biomarker(s) of which the expression level is measured at step a) is a/are protein biomarker(s).

15. System for estimating the effectiveness of treatment with a T-lymphocyte cell co-stimulation modulator agent in a patient with rheumatoid arthritis and who has had an inadequate response to at least one prior treatment with biotherapy, said system comprising:
means for measuring or receiving measurement data of the expression level of at least two biomarkers selected from the group consisting of C4b-Binding Protein (C4BP), C-Reactive Protein (CRP), Cartilage Oligomeric Matrix Protein (COMP), Fibronectin (FN) and Lipopolysaccharide-Binding Protein (LBP) in a biological sample from said patient,
means for processing measurement data configured to estimate said effectiveness of treatment in said patient as a function of each expression level measured for a biomarker selected from this group.
